(19) United States
(12) Patent Application Publication
(10) Pub. No.: US 2024/0390691 A1
(43) Pub. Date: Nov. 28, 2024

(54) WEARABLE ASSEMBLIES FOR TISSUE STIMULATION

(71) Applicant: CURONIX LLC, Pompano Beach, FL (US)

(72) Inventors: Laura Tyler Perryman, Pompano Beach, FL (US); Nick Marinos, Miami, FL (US); Chad David Andresen, Miami Beach, FL (US); Oscar Steven Gil, Plantation, FL (US); William Szatkiewicz, Fort Lauderdale, FL (US); Benjamin Speck, Boca Raton, FL (US); Elizabeth Greene, Pompano Beach, FL (US); Scott Berkeley Sidwell, Fleming Island, FL (US)

(21) Appl. No.: 18/790,636

(22) Filed: Jul. 31, 2024

Related U.S. Application Data
(63) Continuation of application No. 18/093,638, filed on Jan. 5, 2023.
Application No. 18/093,638 is a continuation of application No. 17/157,898, filed on Jan. 25, 2021, now abandoned.
(60) Provisional application No. 62/965,137, filed on Jan. 23, 2020.
(60) Provisional application No. 62/964,933, filed on Jan. 23, 2020.

(51) Int. Cl. A61N 1/378; A61N 1/02; A61N 1/08; A61N 1/36; A61N 1/372

(52) U.S. Cl. CPC A61N 1/3787; A61N 1/36014; A61N 1/37229; A61N 1/025; A61N 2001/083; A61N 1/36071; A61N 1/37247

(57) ABSTRACT

A wearable assembly is configured to generate electrical pulses for transmission to an implanted tissue stimulator. The wearable assembly includes a wearable docking device, a plug-in device configured to mate with the wearable docking device, and a pulse generation module. The pulse generation module includes first internal electronics configured to generate the electrical pulses and located within the wearable docking device or within the plug-in device and second internal electronics providing a power source for the first internal electronics and located within the wearable docking device or within the plug-in device. The wearable assembly further includes a pulse transmission cable for transmitting the electrical pulses to a transmission antenna positioned adjacent the implanted tissue stimulator.

### CROSS-REFERENCE TO RELATED APPLICATIONS

This application is a continuation of U.S. application Ser. No. 18/093,638, filed Jan. 5, 2023, which is a continuation of U.S. application Ser. No. 17/157,898, filed Jan. 25, 2021, which claims the benefit of U.S. Provisional Application No. 62/965, 137, filed on Jan. 23, 2020, and U.S. Provisional Application No. 62/964,933,filed on Jan. 23, 2020. The disclosure of each of the foregoing applications is incorporated herein by reference.

### TECHNICAL FIELD

This disclosure relates to wearable assemblies that are designed to generate electrical pulses for tissue stimulation, such as modular wearable assemblies that include a wearable docking device and a mating plug-in device.

### BACKGROUND

Modulation of tissue within the body by electrical stimulation has become an important type of therapy for treating chronic, disabling conditions, such as chronic pain, problems of movement initiation and control, involuntary movements, dystonia, urinary and fecal incontinence, sexual difficulties, vascular insufficiency, and heart arrhythmia.

For example, an external antenna can be used to send electrical energy to electrodes on an implanted tissue stimulator that can pass pulsatile electrical currents of controllable frequency, pulse width, and amplitudes to a tissue.

### SUMMARY

This disclosure generally relates to modular wearable assemblies that are designed to generate electrical pulses for transmission to an implanted tissue stimulator. In some embodiments, a wearable assembly includes a wearable docking device, a mating plug-in device, and a pulse transmission cable.

In one aspect, a wearable assembly is configured to generate electrical pulses for transmission to an implanted tissue stimulator. The wearable assembly includes a wearable docking device, a plug-in device configured to mate with the wearable docking device, and a pulse generation module. The pulse generation module includes first internal electronics configured to generate the electrical pulses and located within the wearable docking device or within the plug-in device and second internal electronics providing a power source for the first internal electronics and located within the wearable docking device or within the plug-in device. The wearable assembly further includes a pulse transmission cable for transmitting the electrical pulses to a transmission antenna positioned adjacent the implanted tissue stimulator.

In some embodiments, the first internal electronics are contained within the wearable docking device, and the second internal electronics are contained within the plug-in device.

In some embodiments, the first and second internal electronics are contained within the plug-in device.

In some embodiments, the pulse transmission cable is attached to the plug-in device such that the plug-in device comprises a stand-alone device that is operable independently of the wearable docking device.

In some embodiments, the wearable docking device includes a battery and a charging port for the battery.

In some embodiments, the pulse transmission cable is attached to the wearable docking device.

In some embodiments, the wearable docking device includes a clip for grasping a wearable article.

In some embodiments, the wearable assembly further includes a rotary adjustment wheel that is configured to adjust an amplitude of the electrical pulses and that is carried on either the plug-in device or the docking device.

In some embodiments, the wearable docking device includes a sleeve.

In some embodiments, the wearable assembly further includes the transmission antenna, and the pulse transmission cable and the transmission antenna are embedded within the sleeve.

In some embodiments, the wearable docking device further includes a receiving antenna that is embedded within the sleeve and configured to monitor backscatter from the implanted tissue stimulator.

In some embodiments, the wearable docking device further includes multiple skin contacting electrodes that are attached to the sleeve and configured to sense bioelectrical signals and additional internal electronics contained within the sleeve for supporting functionalities of the multiple skin contacting electrodes.

In some embodiments, the additional electronics include one or more of an instrument amplifier, an A/D converter, and a DSP processor and memory.

In some embodiments, the multiple skin contacting electrodes are further configured to deliver transcutaneous stimulation, and the wearable docking device further includes a TENS pulse generator contained within the sleeve.

In some embodiments, the multiple skin contacting electrodes are configured to sense a capacitive load to make a determination as to whether the sleeve is in contact with skin or not in contact with skin, such that either or both of the plug-in device and the docking device are controllable to turn on or turn off automatically.

In some embodiments, the wearable docking device includes additional electronics that implement a non-volatile memory for storing patient data of multiple patients.

In some embodiments, the wearable docking device includes additional electronics that implement a wireless communication module.

In some embodiments, the plug-in device includes additional electronics that implement a wireless communication module.

In some embodiments, the plug-in device includes additional electronics that implement one or more sensors.

In another aspect, a tissue stimulation system includes a wearable assembly configured to generate electrical pulses for transmission to an implanted tissue stimulator, a pulse transmission cable for transmitting the electrical pulses to a transmission antenna, and a tissue stimulator configured to deliver the electrical pulses from the transmission antenna to a tissue. The wearable assembly includes a wearable docking device, a plug-in device configured to mate with the wearable docking device, and a pulse generation module. The pulse generation module includes first internal electronics configured to generate the electrical pulses and located within the wearable docking device or within the plug-in device and second internal electronics providing a power source for the first internal electronics and located within the wearable docking device or within the plug-in device. The wearable assembly further includes a pulse transmission cable for transmitting the electrical pulses to a transmission antenna positioned adjacent the implanted tissue stimulator.

### DESCRIPTION OF DRAWINGS

FIG. 1 is a system block diagram of a tissue stimulation system.

FIG. 2A is a side view of a pulse generator of the tissue stimulation system of FIG. 1, embodied as a wearable module.

FIG. 2B is a side view of the wearable module of FIG. 2A, with a modular enclosure of the wearable module shown as transparent in order to expose internal features of the wearable module.

FIG. 3A is a front view of the wearable module of FIG. 2A.

FIG. 3B is a front view of the wearable module of FIG. 2A, with the modular enclosure of the wearable module shown as transparent in order to expose internal features of the wearable module.

FIG. 4A is a top view of the wearable module of FIG. 2A.

FIG. 4B is a top view of the wearable module of FIG. 2A, with the modular enclosure of the wearable module shown as transparent in order to expose internal features of the wearable module.

FIG. 5A is a bottom view of the wearable module of FIG. 2A.

FIG. 5B is a bottom view of the wearable module of FIG. 2A, with the modular enclosure of the wearable module shown as transparent in order to expose internal features of the wearable module.

FIG. 6 is a side view of a wearable assembly including a docking device formed as a clip, a mating plug-in device, a pulse generation module, and a pulse transmission cable.

FIG. 7 is a side view of a wearable assembly including a pulse generation module that is distributed between a docking device formed as a clip and a mating plug-in device and a pulse transmission cable that extends from the docking device.

FIG. 8 is a side view of a wearable assembly including a docking device formed as a clip, a pulse generation module that is provided in a mating plug-in device, and a pulse transmission cable that extends from the docking device.

FIG. 9 is a side view of a wearable assembly including a docking device formed as a clip with a bridge connector, a pulse generation module that is provided in a mating plug-in device, and a pulse transmission cable that extends from the docking device.

FIG. 10 is a side view of a wearable assembly including a docking device formed as a clip, a pulse generation module that is provided in a mating plug-in device, and a pulse transmission cable that extends from the plug-in device.

FIG. 11 is a front view of a wearable assembly including a docking device formed as a necklace or lanyard, a mating plug-in device, a pulse generation module, and a pulse transmission cable.

FIG. 12 is a perspective view of a wearable assembly including a docking device formed as a sleeve with an embedded transmission antenna, a mating plug-in device, and a pulse generation module distributed between the docking device and the plug-in device.

FIG. 13 is a perspective view of a wearable assembly including a docking device formed as a sleeve with an embedded transmission antenna, along with a pulse generation module provided in a mating plug-in device.

FIG. 14 is a perspective view of a wearable assembly including a docking device formed as a sleeve with an embedded transmission antenna and an embedded receiving antenna, a mating plug-in device, and a pulse generation module distributed between the docking device and the plug-in device.

FIG. 15 is a perspective view of a wearable assembly including a docking device formed as a sleeve with an embedded transmission antenna and skin contacting electrodes, along with a pulse generation module provided in a mating plug-in device.

FIG. 16 is a side view of the wearable assembly of FIG. 15.

FIG. 17 is a perspective view of a wearable assembly including a pulse generation unit that is electrically connected to a sleeve with an embedded transmission antenna.

FIG. 18 is a detailed block diagram of the tissue stimulation system of FIG. 1.

### DETAILED DESCRIPTION

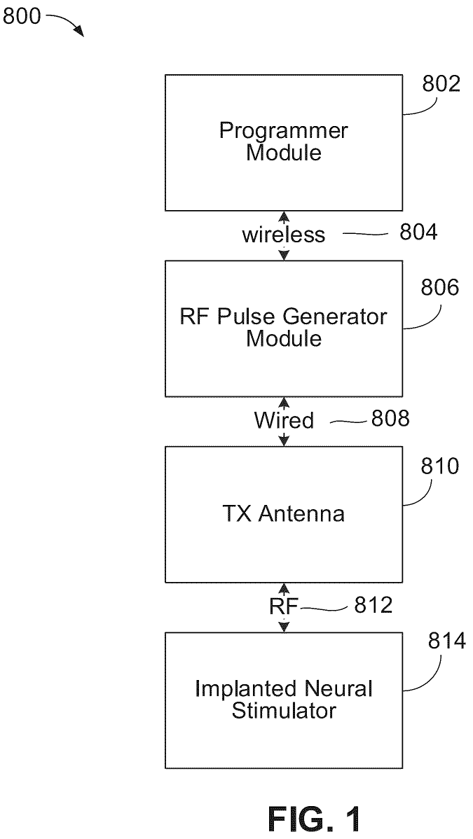

FIG. 1 illustrates a tissue stimulation system 800 (e.g., a neural stimulation system) for delivering electrical therapy to a target tissue within a patient's body. The tissue stimulation system 800 includes a pulse generator 806 that is located exterior to the patient, a transmit (TX) antenna 810 that is connected to the pulse generator 806 and positioned against a skin surface of the patient, a programmer module 802 that runs a software application, and a tissue stimulator 814 this is to be implanted adjacent the target tissue within the body. The tissue stimulation system 800 is designed to send electrical pulses to a nearby (e.g., adjacent or surrounding) target nerve tissue to stimulate the target nerve tissue by using remote radio frequency (RF) energy from TX antenna 810 without cables and without inductive coupling to power the tissue stimulator 814. Accordingly, the tissue stimulator 814 is provided as a passive tissue stimulator in the tissue stimulation system 800. In some examples, the target nerve tissue is in the spinal column and may include one or more of the spinothalamic tracts, the dorsal horn, the dorsal root ganglia, the dorsal roots, the dorsal column fibers, and the peripheral nerves bundles leaving the dorsal column or the brainstem. In some examples, the target nerve tissue may include one or more of cranial nerves, abdominal nerves, thoracic nerves, trigeminal ganglia nerves, nerve bundles of the cerebral cortex, deep brain, sensory nerves, and motor nerves. In other words, targets may be in the central and/or peripheral nervous system.

In some embodiments, the software application supports a wireless connection 804 (e.g., via Bluetooth®). The software application can enable the user to view a system status and system diagnostics, change various parameters, increase and decrease a desired stimulus amplitude of the electrical pulses, and adjust a feedback sensitivity of the RF pulse generator module 806, among other functions.

The RF pulse generator module 806 includes stimulation circuitry, a battery to power generator electronics, and communication electronics that support the wireless connection 804. In some embodiments, the RF pulse generator module 806 is designed to be worn external to the body, and the TX antenna 810 (e.g., located external to the body) is connected to the RF pulse generator module 806 by a wired connection 808. Accordingly, the RF pulse generator module 806 and/or the TX antenna 810 may be incorporated into a wearable accessory (e.g., a belt or a harness design) or a clothing article such that electric radiative coupling can occur through the skin and underlying tissue to transfer power and/or control parameters to the tissue stimulator 814.

The TX antenna 810 can be coupled directly to tissues within the body to create an electric field that powers the implanted tissue stimulator 814. The TX antenna 810 communicates with the tissue stimulator 814 through an RF interface. For instance, the TX antenna 810 radiates an RF transmission signal that is modulated and encoded by the RF pulse generator module 806. The tissue stimulator 814 includes one or more antennas (e.g., dipole antennas) that can receive and transmit through an RF interface 812. In particular, the coupling mechanism between the TX antenna 810 and the one or more antennas on the tissue stimulator 814 is electrical radiative coupling and not inductive coupling. In other words, the coupling is through an electric field rather than through a magnetic field. Through this electrical radiative coupling, the TX antenna 810 can provide an input signal to the tissue stimulator 814.

In addition to the one or more antennas, the tissue stimulator 814 further includes internal receiver circuit components that can capture the energy carried by the input signal sent from the TX antenna 810 and demodulate the input signal to convert the energy to an electrical waveform. The receiver circuit components can further modify the waveform to create electrical pulses suitable for stimulating the target neural tissue. The tissue stimulator 814 further includes electrodes that can deliver the electrical pulses to the target neural tissue. For example, the circuit components may include wave conditioning circuitry that rectifies the received RF signal (e.g., using a diode rectifier), transforms the RF energy to a signal suitable for the stimulation of neural tissue, and presents the resulting waveform to one or more electrodes. In some implementations, the power level of the input signal directly determines an amplitude (e.g., a power, a current, and/or a voltage) of the electrical pulses applied to the target neural tissue by the electrodes. For example, the input signal may include information encoding stimulus waveforms to be applied at the electrodes.

In some implementations, the RF pulse generator module 806 can remotely control stimulus parameters of the electrical pulses applied to the target neural tissue by the electrodes and, in some embodiments, may also monitor feedback from the tissue stimulator 814 based on RF signals received from the tissue stimulator 814. For example, a feedback detection algorithm implemented by the RF pulse generator module 806 can monitor data sent wirelessly from the tissue stimulator 814, including information about the energy that the tissue stimulator 814 is receiving from the RF pulse generator 806 and information about the stimulus waveform being delivered to the electrodes. Accordingly, the circuit components internal to the tissue stimulator 814 may also include circuitry for communicating information back to the RF pulse generator module 806 to facilitate the feedback control mechanism. For example, the tissue stimulator 814 may send to the RF pulse generator module 806 a stimulus feedback signal that is indicative of parameters of the electrical pulses, and the RF pulse generator module 806 may employ the stimulus feedback signal to adjust parameters of the signal sent to the tissue stimulator 814.

In order to provide an effective therapy for a given medical condition, the tissue stimulation system 800 can be tuned to provide the optimal amount of excitation or inhibition to the nerve fibers by electrical stimulation. A closed loop feedback control method can be used in which the output signals from the tissue stimulator 814 are monitored and used to determine the appropriate level of neural stimulation current for maintaining effective neuronal activation. Alternatively, in some cases, the patient can manually adjust the output signals in an open loop control method.

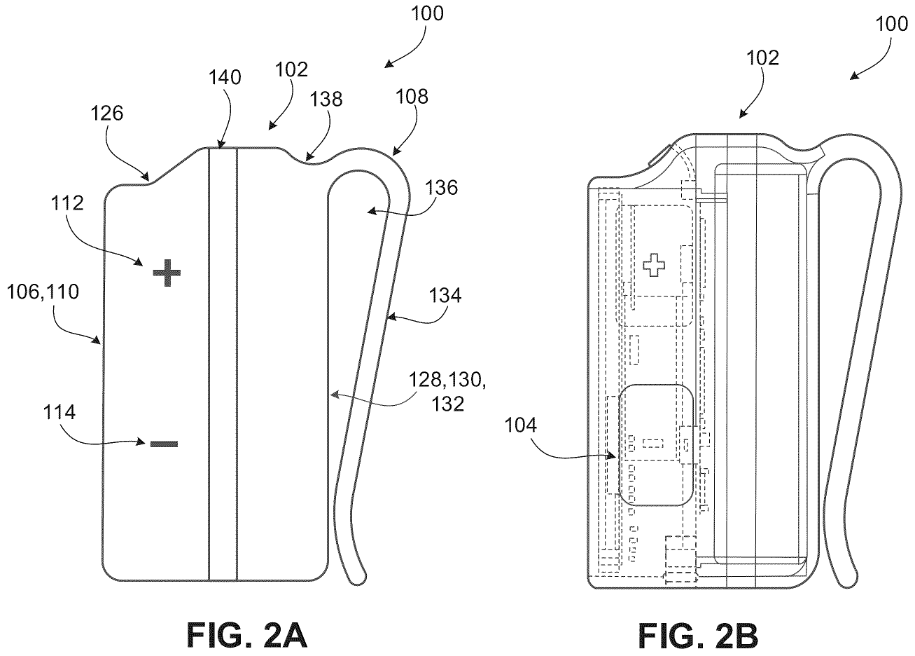

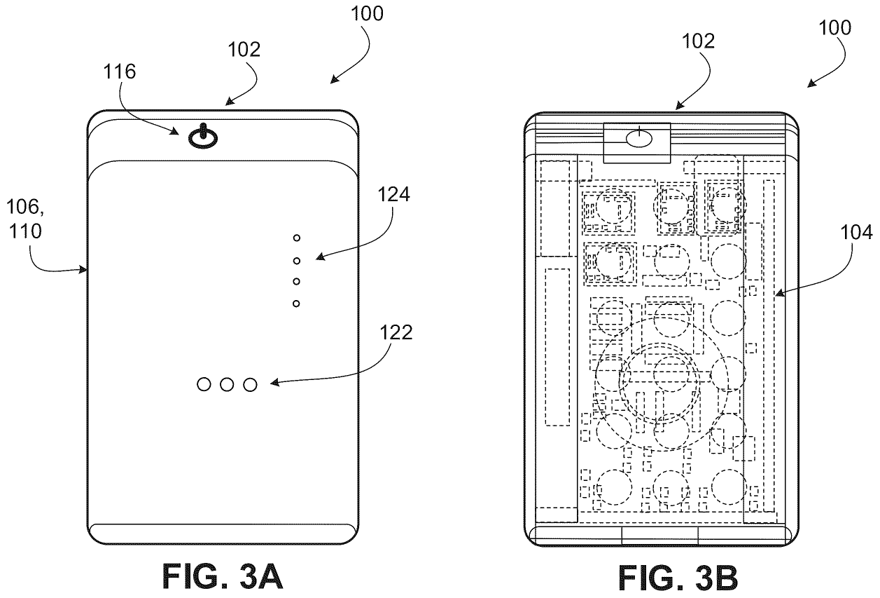

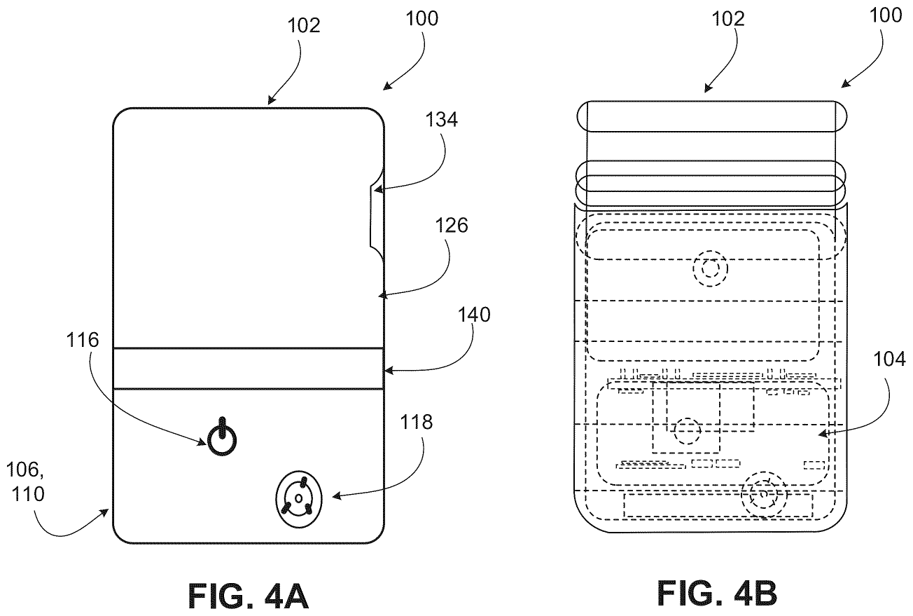

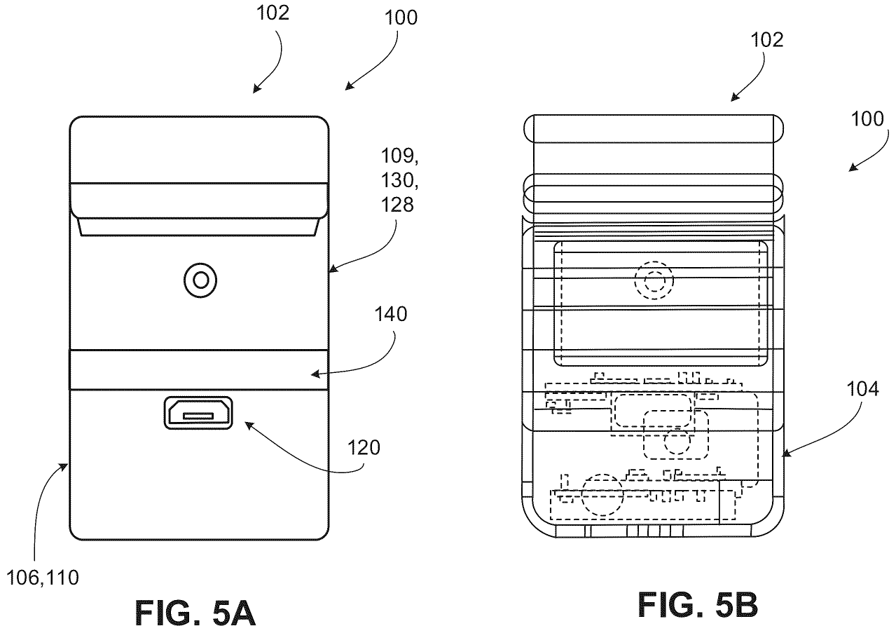

FIGS. 2A-5B illustrate various views of an example embodiment of the RF pulse generator module 806 provided as a wearable module 100 that can be secured to an article of clothing. The wearable module 100 includes a modular enclosure 102 and internal circuitry 104 housed within the modular enclosure 102. The modular enclosure 102 includes a transmission unit 106 (e.g., a plug-in device), a power unit 108 (e.g., a wearable docking device), and an interface section 140 that couples the transmission unit 106 to the power unit 108. The wearable module 100 is a lightweight, compact device that is easily manipulated by a user. For example, in some embodiments, the wearable module 100 has a weight in a range of about 0.1 lbs to about 0.3 lbs. In some embodiments, the wearable module 100 has a total height in a range of about 5.0 cm to about 8.0 cm, a total thickness in a range of about 2.5 cm to about 5.0 cm, and a total width in a range of about 2.0 cm to about 3.0 cm. Furthermore, the wearable module 100 has generally rounded edges for comfortable gripping of the wearable module 100 by hand.

The transmission unit 106 includes the internal circuitry 104 and a housing 110 that surrounds the internal circuitry 104. The housing 110 provides selectors 112, 114 (e.g., buttons) that can be pressed by a user to control (e.g., increase or decrease) the amplitude of electrical pulses delivered to the target tissue. In some embodiments, the transmission unit 106 may alternatively include internal force sensors and associated external selectors that can merely be tapped for adjustment instead of being pressed for adjustment. In some embodiments, other adjustment means may be used, such as knobs, sliders, rotary wheel(s), etc. or other adjustment means that rely on haptic feedback. In some embodiments, the selectors 112, 114 may be eliminated altogether in lieu of using a capacitive, IR, or inductive sensor to sense whether the device is on or off and to automatically turn the system off or on based on this sensing. In some embodiments, security measures such as a fingerprint or other authorization may identify a patient, implement the patient's preferred stimulation parameters, and/or be required to adjust stimulation parameters, e.g., the amplitude of electrical pulses. The housing 110 also provides a power button 116 for turning the wearable module 100 on and off and a port 118 (e.g., a recessed port) for connecting the wearable module 100 to the TX antenna 810 via the wired connection 808 (e.g., a cable). Additionally, the housing 110 may provide a port 120 (e.g., a micro-USB port) for charging and/or running diagnostics on the wearable module 100. The housing 110 may further include indicators 122, 124 (e.g., LED indicators) that are programmable to indicate one or more statuses, such as battery life or a strength of the pulse amplitude.

The housing 110 has a generally rectangular cross-sectional shape with a curved surface profile 126 along a top edge. In some embodiments, the housing 110 is made of one or more materials, such as hardened plastics or other plastics (e.g., acrylonitrile butadiene styrene (ABS)) and metals (e.g., aluminum or steel). For example, in some embodiments, the housing 110 includes an outer plastic housing and an inner RF cage or an inner plastic cage that is coated with a spray that can act as an RF cage.

The power unit 108 includes a battery 128 (e.g., a rechargeable battery) and a housing 130 that surrounds the battery 128. The housing 130 includes a main body 132 and an integral clip 134 that extends from the main body 132. The clip 134 is designed to be placed over a portion of an article of clothing to secure the wearable module 100 to the article of clothing. For example, the housing 130 defines a holding region 136 in which the portion of clothing can be retained between the body 132 and the clip 134. Accordingly, the clip 134 is flexible enough to be pulled away from the body 132 for grasping and release of the portion of clothing. The power unit 108 is detachable from the transmission unit 106 at the interface section 140 so that the battery 128 contained in power unit 108 can be recharged (e.g., at a mating charging station). The main body 132 of the housing 130 has a generally rectangular cross-sectional shape with a curved surface profile 138 along a top edge that transitions to the clip 134. In some embodiments, the housing 130 is made of one or more materials, such as plastics (e.g., ABS) and metals (e.g., in the form of an inner metal frame that provides structural support).

Additional features of the wearable module 100 may include an LCD or LED interface (e.g., an E-Ink display), automatic shutoff to prevent overcharging of the power unit 108, piezo and volume control for sound and interface, an internal gyroscope, magnetometer, and accelerometer for optimizing stimulation based on patient health data, and power protection circuitry to protect from surging of the RF (e.g., a safety circuit).

While the wearable module 100 has been described and illustrated with respect to certain dimensions, sizes, shapes, arrangements, and materials, in some embodiments, a wearable module that is otherwise substantially similar in construction and function to the wearable module 100 may include one or more different dimensions, sizes, shapes, arrangements, and materials.

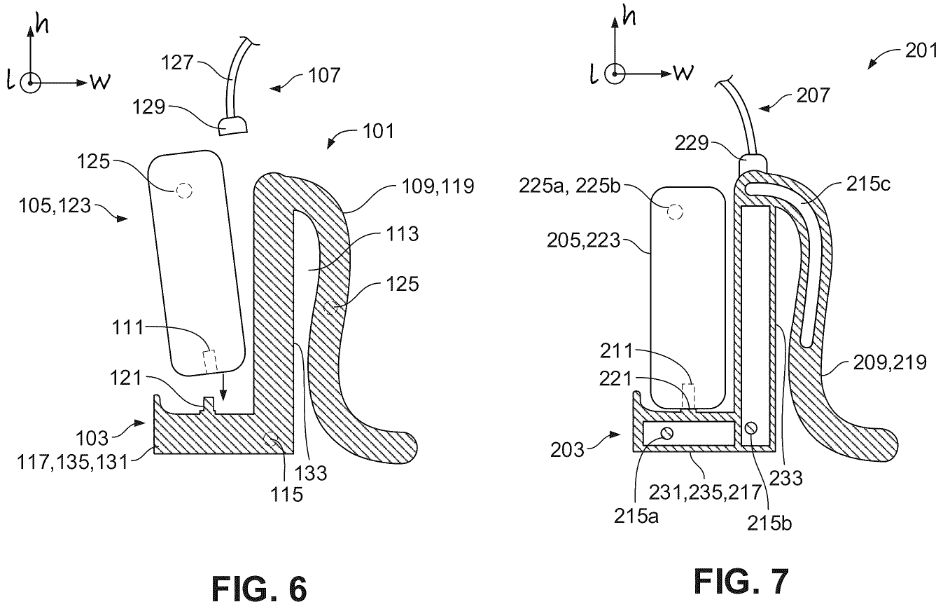

FIG. 6 illustrates an example embodiment of a wearable assembly 101 that can be secured to a portion of a wearable article. For example, the wearable assembly 101 may be secured to a pocket, a loop, or another portion or piece of clothing or to a portion of a wearable accessory, such as a belt. The wearable assembly 101 has a modular design that is provided by a docking device 103 (e.g., a first module), a cooperating plug-in device 105 (e.g., a second module), and an RF cable 107. In some embodiments, the docking device 103 has a total width (along an axis w) in a range of about 0.5 cm to about 4 cm, a total length (along an axis l) in a range of about 2 cm to about 8 cm, and a total height (along an axis h) in a range of about 2 cm to about 8 cm. In some embodiments, the plug-in device 105 has a width (along an axis w) in a range of about 0.4 cm to about 4cm, a length (along an axis l) in a range of about 1.5 cm to about 7 cm, and a height (along an axis h) in a range of about 1.5 cm to about 7 cm. The plug-in device 105 can be attached to and removed from (e.g., pulled from) the docking device 103 as desired for administering a tissue stimulation treatment. In some embodiments, the docking device 103 and the plug-in device 105 together have a total weight of about 0.05 kg to about 1kg.

The docking device 103 (e.g., a docking station or a dock) includes internal electronics 115 that support various functionalities of the wearable assembly 101. The docking device 103 also includes a rigid receptacle 135 that supports the plug-in device 105 and a flexible clip 109 that extends from the receptacle 135. The internal electronics 115 may be contained in either or both of the receptacle 135 (e.g., within a base 131 and/or a wall 133) and the clip 109, depending on a configuration of the wearable assembly 101, as will be discussed in more detail below. The receptacle 135 includes a housing 117 that is generally L-shaped in cross-section and that may contain all or a portion of the internal electronics 115. The receptacle 135 is also equipped with a connector 121 for electrical connection to a port 111 of the plug-in device 105.

The receptacle 135 and the clip 109 together form a holding region 113 in which the wearable article can be retained by the docking device 103. Accordingly, the clip 109 is flexible enough to be pulled away from the receptacle 135 for grasping and release of the wearable article. The clip 109 includes a housing 119 that has a curved cross-sectional shape and that may contain all or a portion of the internal electronics 115. The housings 117, 119 may be integral with each other to form a single housing structure or may be provided as separate components that are attached to each other. In some embodiments, the housings 117, 119 are made of one or more of polycarbonate, ABS, silicone, carbon fiber, or aluminum.

In addition to the port 111, the plug-in device 105 also includes a housing 123 and internal electronics 125 for supporting various functionalities of the wearable assembly 101. The housing 123 of the plug-in device 103 has a generally rectangular cross-sectional shape and has smooth, rounded edges for comfortable handling by a user. In some embodiments, the housing 123 is made of one or more of polycarbonate, ABS, silicone, carbon fiber, or aluminum.

The RF pulse generator module 806 of the tissue stimulation system 800 may be implemented by one or both of the internal electronics 115 of the docking device 103 and the internal electronics 125 of the plug-in device 105, depending on a configuration of the wearable assembly 101. Furthermore, the wired connection 808 of the tissue stimulation system 800 may be embodied as the RF cable 107. The RF cable 107 includes a cable shaft 127 and a connector 129 by which the RF cable 107 may be connected to either the docking device 103 or the plug-in device 105, depending on a configuration of the wearable assembly 101. Accordingly, an opposite end of the RF cable 107 may be attached to the TX antenna 810 of the tissue stimulation system 800. Owing to a variety of configurations that can result from an implementation of the RF pulse generator module 806 within the docking device 103 or the plug-in device 105 and to a connection site of the RF cable 107, the wearable assembly 101 may be embodied as any of the wearable assemblies 201, 301, 401, 501 that will be discussed below with respect to FIGS. 7-10.

FIG. 7 illustrates a wearable assembly 201 that includes a docking device 203, a plug-in device 205, and an RF cable 207 that is connected at a connector 229 to a position (e.g., the top of rigid receptacle 235) on the docking device 203. The wired connection 808 of the tissue stimulation system 800 may be embodied as the RF cable 207, and the RF cable 207 may be connected to the TX antenna 810 of the tissue stimulation system 800 at an opposite end. The docking device 203 contains internal electronics 215a, 215b, 215c within a housing 217 of the receptacle 235 and within a housing 219 of a flexible clip 209 of the docking station 203. The receptacle 235 is also equipped with a connector 221 for electrical connection to a port 211 of the plug-in device 205. The flexible clip 209 extends from the receptacle 235 to form a holding region 213 for grasping and release of a wearable article. In addition to the port 211, the plug-in device 205 also includes a housing 223 and internal electronics 225a, 225b contained within the housing 223.

In the wearable assembly 201, the RF pulse generation functionalities of the RF pulse generator module 806 of the tissue stimulation system 800 are provided as part of the docking device 203. For example, the internal electronics 215a may be implemented as an RF synthesizer that is located within a base 231 of the housing 217 of the receptacle 235, while the internal electronics 215b may be implemented as ancillary RF components that are located within the housing 219 of the clip 209. The internal electronics 215c are located within a wall 233 of the housing 217 of the receptacle 235 and may be implemented as a main stage gain amplifier for amplifying electrical pulses generated by the RF synthesizer.

With the RF pulse generation functionalities of the RF pulse generator 806 contained within the docking device 203, the plug-in device 205 is provided as a digital battery pack that provides the powering feature of the RF pulse generator module 806. For example, the internal electronics 225a, 225b of the plug-in device 205 may be implemented respectively as a battery and a controller for powering and controlling the internal electronics 215a, 215b, 215c of the docking device 203. Accordingly, the connector 221 of the docking device 203 is provided as a power and data connector. RF components are often relatively large. Therefore, distribution of the internal electronics 215a, 215b, 215c entirely within and across the docking device 203 allow the plug-in device 205 to be formed with an overall small size and thin profile for comfortable handling at a relatively low cost due to exclusion of larger RF components. Such a low cost may enable a patient to purchase multiple plug-in devices 205 that can all be used with the same docking device 203. In some embodiments, the plug-in device 205 has width (along an axis w) in a range of about 0.4 cm to about 4 cm, a length (along an axis l) in a range of about 1.5 cm to about 7 cm, and a height (along an axis h) in a range of about 1.5 cm to about 7 cm.

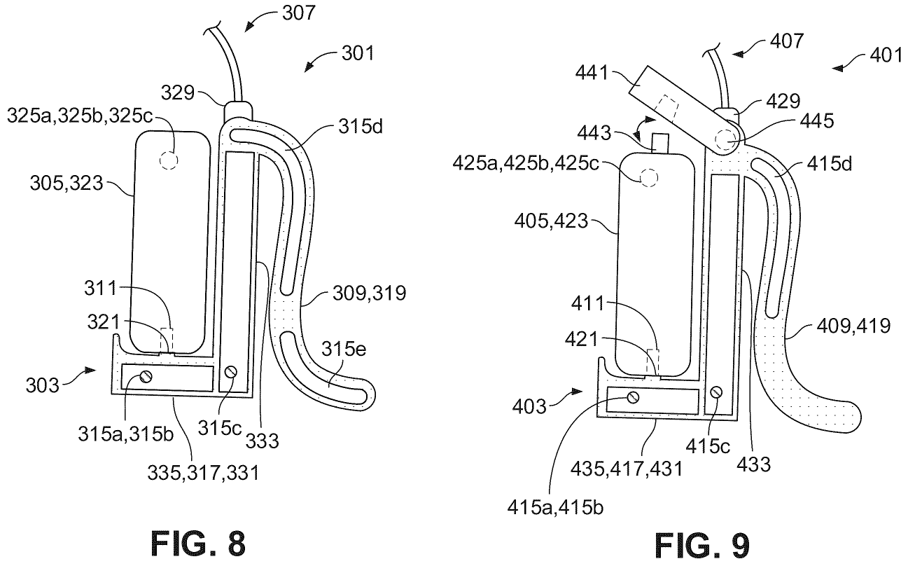

FIG. 8 illustrates a wearable assembly 301 that includes a docking device 303, a plug-in device 305, and an RF cable 307 that is connected at a connector 329 to a location (e.g., the top of rigid receptacle 335) on the docking device 303. The wired connection 808 of the tissue stimulation system 800 may be embodied as the RF cable 307, and the RF cable 307 may be connected to the TX antenna 810 of the tissue stimulation system 800 at an opposite end. The docking device 303 contains internal electronics 315a, 315b, 315c, 315d, 315e within a housing 317 of the receptacle 335 and within a housing 319 of a flexible clip 309 of the docking station 303. The receptacle 335 is also equipped with a connector 321 for electrical connection to a port 311 of the plug-in device 305. The clip 309 extends from the receptacle 335 to form a holding region 313 for grasping and release of a wearable article. In addition to the port 311, the plug-in device 305 includes a housing 323 and internal electronics 325a, 325b, 325c contained within the housing 323.

In the wearable assembly 301, the RF pulse generator module 806 of the tissue stimulation system 800 is provided as part of the plug-in device 305. For example, the internal electronics 325a may be implemented as an RF source, while the internal electronics 325b, 325c may be implemented respectively as a battery and a controller for powering and controlling the internal electronics 325a of the plug-in device 305 and the internal electronics 315a, 315b, 315c, 315d, 315e of the docking device 303. Accordingly, the connector 321 of the docking device 303 is provided as a power, data, and RF connector. While the RF pulse generator module 806 is contained entirely within the plug-in device 305, the docking device 303 provides additional functionalities.

The internal electronics 315c are located within a wall 333 of the housing 317 of the receptacle 335 and may be implemented as a second stage gain amplifier that boosts RF power output by the plug-in device 305 (e.g., by up to 100 W). The internal electronics 315a, 315b are located within a base 331 of the housing 317. The internal electronics 315a may be implemented as a secondary processor for controlling the internal electronics 315c, while the internal electronics 315b may be implemented as a non-volatile memory for storing unique patient data (e.g., patient IDs and patient health parameters) for multiple patients. Storage of patient data for multiple patients can allow the docking device 303 to be used interchangeably with multiple plug-in devices 305 that are associated with multiple patients. The internal electronics 315d, located within the housing 319 of the clip 309, may be implemented as a wireless communication module that can optionally allow wireless communication (e.g., via a WiFi network, a Zigbee network, or another local area network) between the plug-in device 305 and the TX antenna 810 of the tissue stimulation system 800 without use of the RF cable 307. The internal electronics 315e, also located within the housing 319, may be implemented as one or more sensors (e.g., electrical sensors, mechanical sensors, MEMS sensors, etc.) for determining an orientation of the docking device 305 or for recording a velocity, a health indicator, or another parameter associated with a patient's physical activity or health.

FIG. 9 illustrates a wearable assembly 401 that includes a docking device 403, a plug-in device 405, and an RF cable 407 that is connected at a connector 429 to a position (e.g., the top of rigid receptacle 435) on the docking device 403. The RF cable 407 may be connected to the TX antenna 810 of the tissue stimulation system 800 at an opposite end. The docking device 403 contains internal electronics 415a, 415b, 415c, 415d within a housing 417 of the receptacle 435 and within a housing 419 of a flexible clip 409 of the docking station 403. The receptacle 435 is also equipped with a connector 421 for electrical connection to a port 411 of the plug-in device 405 and with a hinged bridge connector 441 for electrical connection to a connector 443 at an opposite, upper end of the plug-in device 405. The clip 409 extends from the receptacle 435 to form a holding region 413 for grasping and release of a wearable article. In addition to the port 411 and the RF connector 443, the plug-in device 405 also includes a housing 423 and internal electronics 425a, 425b, 425c contained within the housing 423.

In the wearable assembly 401, the RF pulse generator module 806 of the tissue stimulation system 800 is provided as part of the plug-in device 405. For example, the internal electronics 425a may be implemented as an RF source, while the internal electronics 425b, 425c may be implemented respectively as a battery and a controller for powering and controlling the internal electronics 425a of the plug-in device 405 and the internal electronics 415a, 415b, 415c, 415d of the docking device 403. Accordingly, the connector 421 of the docking device 403 is provided as a power and data connector, while the connectors 441, 443 of the plug-in device 405 and of the docking device 403 are provided as RF connectors. The bridge connector 441 is pivotable at a hinge 445 of the housing 417 of the receptacle 435 to allow unobstructed docking and removal of the plug-in device 405 within the receptacle 435. While the RF pulse generator module 806 is contained entirely within the plug-in device 405, the docking device 403 provides additional functionalities.

The internal electronics 415c are located within a wall 433 of the housing 417 of the receptacle 435 and may be implemented as a second stage gain amplifier that boosts RF power output by the plug-in device 405 (e.g., by up to 100 W). The internal electronics 415a, 415b are located within a base 431 of the housing 417. The internal electronics 415a may be implemented as a secondary processor for controlling the internal electronics 415c, while the internal electronics 415b may be implemented as a non-volatile memory for storing unique patient data for multiple patients to allow the docking device 403 to be used interchangeably with multiple plug-in devices 405 that are associated with multiple patients. The internal electronics 415d, located within the housing 419 of the clip 409, may be implemented as a wireless communication module that can optionally allow wireless communication between the plug-in device 405 and the TX antenna 810 of the tissue stimulation system 800 without use of the RF cable 407.

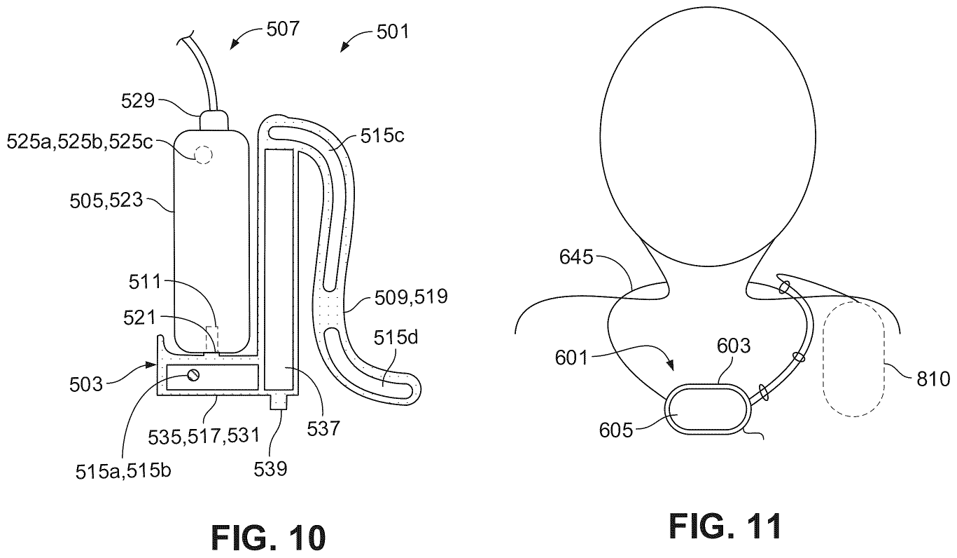

FIG. 10 illustrates a wearable assembly 501 that includes a docking device 503, a plug-in device 505, and an RF cable 507 that is connected at a connector 529 to the plug-in device 505. The RF cable 507 may be connected to the TX antenna 810 of the tissue stimulation system 800 at an opposite end. The docking device 503 contains internal electronics 515a, 515b, 515c, 515d within a housing 517 of a rigid receptacle 535 of the docking station 503 and within a housing 519 of a flexible clip 509 of the docking station 503. The receptacle 535 is also equipped with a battery 537, a charging port 539 for recharging the battery 537, and a connector 521 for electrical connection to a port 511 of the plug-in device 505. The clip 509 extends from the receptacle 535 to form a holding region 513 for grasping and release of a wearable article. In addition to the port 511, the plug-in device 505 also includes a housing 523 and internal electronics 525a, 525b, 525c contained within the housing 523.

In the wearable assembly 501, the RF pulse generator module 806 of the tissue stimulation system 800 is provided as part of the plug-in device 505. For example, the internal electronics 525a may be implemented as an RF source, while the internal electronics 525b, 525c may be implemented respectively as a battery and a controller for powering and controlling the internal electronics 525a of the plug-in device 505 and/or the internal electronics 515a, 515b, 515c, 515d of the docking device 503. Accordingly, the connector 521 of the docking device 503 is provided as a power, data, and RF connector. Since the RF pulse generator module 806 is contained entirely within the plug-in device 505 and since the RF cable 507 is connected directly to the plug-in device 505, the plug-in device 505 is a stand-alone device that is capable of operating independently of the docking device 503. However, the docking device 303 provides additional functionalities.

The battery 537 is located within a wall 533 of the housing 517 of the receptacle 535 and provides advanced/additional powering that can boost power output by the plug-in device 505 for a period of up to about 24 h. The internal electronics 515a, 515b are located within a base 531 of the housing 517. The internal electronics 515a may be implemented as a secondary processor for controlling the internal electronics 515c, while the internal electronics 515b may be implemented as a non-volatile memory for storing unique patient data for multiple patients to allow the docking device 503 to be used interchangeably with multiple plug-in devices 505 that are associated with multiple patients. The internal electronics 515c, located within the housing 519 of the clip 509, may be implemented as a wireless communication module that can optionally allow wireless communication between the plug-in device 505 and the TX antenna 810 of the tissue stimulation system 800 without use of the RF cable 507. The internal electronics 515d, also located within the housing 519, may be implemented as one or more sensors (e.g., for determining an orientation of the docking device 505).

While the wearable assemblies 101, 201, 301, 401, 501 have been described and illustrated with respect to certain dimensions, sizes, shapes, arrangements, and materials, in some embodiments, a wearable assembly that is otherwise substantially similar in construction and function to any of the wearable assemblies 101, 201, 301, 401, 501 may include one or more different dimensions, sizes, shapes, arrangements, and materials. Therefore, other embodiments are possible. For example, while the above-discussed clips 109, 209, 309, 409, 509 of the docking devices 103, 203, 303, 403, 503 have been described and illustrated as having a curved S-shape with certain arrangements of internal electronics, in some embodiments, a wearable assembly may include a docking station with a clip that has a different shape (e.g., a straight, flat shape) with a different arrangement of internal electronics. While the above-discussed receptacles 135, 235, 335, 435, 535 of the docking devices 103, 203, 303, 403, 503 have been described and illustrated as being L-shaped with certain arrangements of internal electronics, in some embodiments, a wearable assembly may include a docking station with a receptacle that has a different shape with a different arrangement of internal electronics and a corresponding plug-in device that also has a different, complementary shape.

For example, FIG. 11 illustrates a wearable assembly 601 that can be secured to a lanyard or necklace 645 worn by a patient. The wearable assembly 601 may be substantially similar in functional capabilities to any of the wearable assemblies 101, 201, 301, 401, 501, but includes a rigid receptacle of a docking device 603 and a cooperating plug-in device 605. The wearable assembly 601 may have a generally oval shape, as shown in FIG. 11, or other desired shape. As in the earlier embodiments, the RF pulse generator module 806 of the tissue stimulation system 800 may be provided as part of the docking device 603 and/or the plug-in device 605. A flexible rear clip (not visible) of the docking device 603 extends from the receptacle to form a holding region for grasping and release of the lanyard or necklace 645. Other attachment mechanisms are possible, such as clamps or hook-and-loop or snaps or other fasteners. Alternatively, the docking device 603 may be permanently connected to the lanyard or necklace 645.

The wearable assembly 601 also includes an RF cable 607 (e.g., an implementation of the wired connection 808 of the tissue stimulation system 800) that extends from either the docking device 603 or the plug-in device 605 to the TX antenna 810 of the tissue stimulation system 800, which is positioned adjacent to the patient's skin above the nerve(s) being stimulated by the implanted neural stimulator 814 of the tissue stimulation system 800. For instance, any of the wearable assemblies 101, 201, 301, 401, 501, 601 may be connected by RF cable or wireless communication as described above (e.g., WiFi, Zigbee, or other local area network) to a TX antenna 810 located as needed to transfer power and data to an implanted neural stimulator 814 positioned with its one or more electrodes adjacent any central or peripheral nervous system nerve(s), as desired.

In some embodiments, any of the above-discussed docking devices 103, 203, 303, 403, 503, 603 and the plug-in devices 105, 205, 305, 405, 505, 605 may include buttons, selectors, or other adjustment means (e.g., any of the adjustment means discussed above with respect to the wearable module 100) for adjusting the amplitude or other parameters of electrical pulses delivered to a target tissue. In some embodiments, such selectors may be eliminated altogether in lieu of using a capacitive, IR, or inductive sensor located at the plug-in device or the docking device to sense whether the device is on or off and to automatically turn the device off or on based on this sensing. In some embodiments, security measures such as a fingerprint or other authorization may identify a patient, implement their preferred stimulation parameters, and/or be required to adjust stimulation parameters, e.g., the amplitude of electrical pulses.

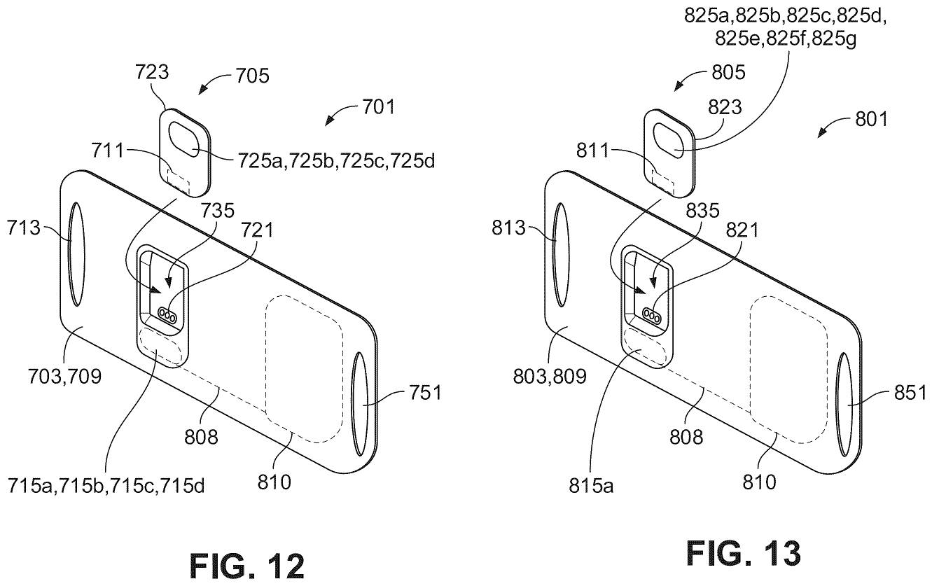

In some embodiments, a wearable assembly may include a docking device that is embedded with the TX antenna 810 and the wired connection 808 of the tissue stimulation system 800. For example, FIG. 12 illustrates such a wearable assembly 701. The wearable assembly 701 has a modular design that is provided by a docking device 703 (e.g., a first module) and a cooperating plug-in device 705 (e.g., a second module). The plug-in device 705 can be attached to and removed from the docking device 703 as desired for administering treatment. In this and subsequent embodiments, a TX antenna 810, instead of being embedded in the sleeve of the docking device, may be a discrete device that is slipped into a pocket in the sleeve of the docking device and may communicate with the wearable assembly via an external wired connection 808 and/or via wireless connection as described above, rather than an embedded connection, as sown in FIG. 12.

Returning to the example shown in FIG. 12, docking device 703 (e.g., a docking station or a dock) includes a flexible fabric sleeve 709 and several components contained within or supported by the sleeve 709. The sleeve 709 can be wrapped snuggly around a patient's body part (e.g., the patient's leg, arm, shoulder, or abdomen) as a compression sleeve. Accordingly, the sleeve 709 is equipped with mating fastening features 713, 751 that are located along opposite edges of the sleeve 709. Example fastening features 713, 751 include hook and loop materials and snap-fit buttons and receptacles. The sleeve 709 may be formed of one or more material layers that provide comfort against the patient's body and may also include antimicrobial properties. Example materials from which the sleeve 709 may be made include neoprene, cotton, silk, polyester, spandex, silicone, and polyurethane.

Within the sleeve 709, the docking device 703 further includes pulse generation functionalities of the RF generator module 806, the wired connection 808, and the TX antenna 810 of the tissue stimulation system 800 as embedded components. The sleeve 709 also contains internal electronics 715a, 715b, 715c, 715d that support various functionalities of the wearable assembly 101. In some embodiments, the internal electronics may be formed on one or more flex circuits for imparting additional flexibility to the docking device 703. The pulse generation functionalities of the RF generator module 806 are provided by the internal electronics 715a, 715b, which may be implemented respectively as an RF synthesizer and an RF gain amplifier for amplifying electrical pulses generated by the RF synthesizer. The internal electronics 715c may be implemented as one or more power detectors for detecting power from the plug-in device 705, while the internal electronics 715d may be implemented as a non-volatile memory for storing unique patient data (e.g., patient IDs and patient health parameters) for multiple patients. Storage of patient data for multiple patients can allow the docking device 703 to be used interchangeably with multiple plug-in devices 705 that are associated with multiple patients.

The sleeve 709 is also equipped with a receptacle 735 that supports the plug-in device 705 at a connector terminal 721. The connection 808 of the tissue stimulation system 800 extends from the TX antenna 810 and terminates at the internal electronics 715a, 715b, 715c, 715d. The internal electronics 715a, 715b, 715c, 715d are also electrically connected to the connector terminal 721. The connector terminal 721 is designed to mate with a connector terminal 711 of the plug-in device 705.

With the RF pulse generation functionalities of the RF pulse generator module 806 contained entirely within the docking device 703, the plug-in device 705 includes a power source of the RF pulse generator module 806. For example, in addition to the connector terminal 711, the plug-in device 705 also includes a housing 723 that contains internal electronics 725a. The internal electronics 725a are implemented as a battery for powering the internal electronics 715a, 715b, 715c, 715d within the docking device 703 and for powering additional internal electronics 725b, 725c, 725d contained within the housing 723. The internal electronics 725b, 725c, 725d may be implemented respectively as one or more processors, a user interface (UI) controller, and the wireless connection 804 of the tissue stimulation system 800. For example, the internal electronics 725d provide a wireless communication module that communicates (e.g., via a WiFi network, a Zigbee network, or another local area network) with the programmer module 802 of the tissue stimulation system 800. Accordingly, the connector terminal 721 of the receptacle 735 provides power and data connections.

The wearable assembly 701 has been described and illustrated as splitting the RF pulse generation functionalities and the RF powering functionality of the RF pulse generator module 806 respectively between the docking device 703 and the plug-in device. However, in some embodiments, a wearable assembly that is otherwise substantially similar in construction and function to the wearable assembly 701 may alternatively include the capabilities of the RF pulse generator module 808 entirely within a plug-in device. For example, FIG. 13 illustrates such a wearable assembly 801. The wearable assembly 801 includes a docking device 803 and a cooperating plug-in device 805 that can be attached to and removed from the docking device 803 as desired for administering treatment.

The docking device 803 includes a flexible fabric sleeve 809 that is substantially similar in construction and function to the sleeve 709 of the wearable assembly 701. The sleeve 809 is accordingly equipped with mating fastening features 813, 851 that are located along opposite edges of the sleeve 809. Within the sleeve 809, the docking device 803 further includes the connection 808 and the TX antenna 810 of the tissue stimulation system 800, e.g., as embedded components. The sleeve also contains internal electronics 815a that may be implemented as a non-volatile memory for storing unique patient data for multiple patients to allow the docking device 803 to be used interchangeably with multiple plug-in devices 805 that are associated with multiple patients.

The sleeve 809 is also equipped with a receptacle 835 that supports the plug-in device 805 at a connector terminal 821. The connection 808 of the tissue stimulation system 800 extends from the TX antenna 810 and terminates at the internal electronics 815a. The connector terminal 821 is designed to mate with a connector terminal 811 of the plug-in device 805.

In the wearable assembly 801, the RF pulse generator module 806 is contained entirely within the plug-in device 805. For example, in addition to the connector terminal 811, the plug-in device 805 also includes a housing 823 that contains internal electronics 825a, 825b, 825c, 825d, 825e, 825f, 825g. The internal electronics 825a, 825b, 825c are implemented respectively as an RF synthesizer, an RF gain amplifier, and a battery for powering the RF pulse generation functionalities. The internal electronics 825c also powers additional internal electronics 825d, 825e, 825f, 825g contained within the housing 823. The internal electronics 825d, 825c, 825f, 825g may be implemented respectively as one or more processors, a UI controller, the wireless connection 804 of the tissue stimulation system 800, and one or more power detectors. Accordingly, the connector terminal 821 of the receptacle 835 provides RF and data connections.

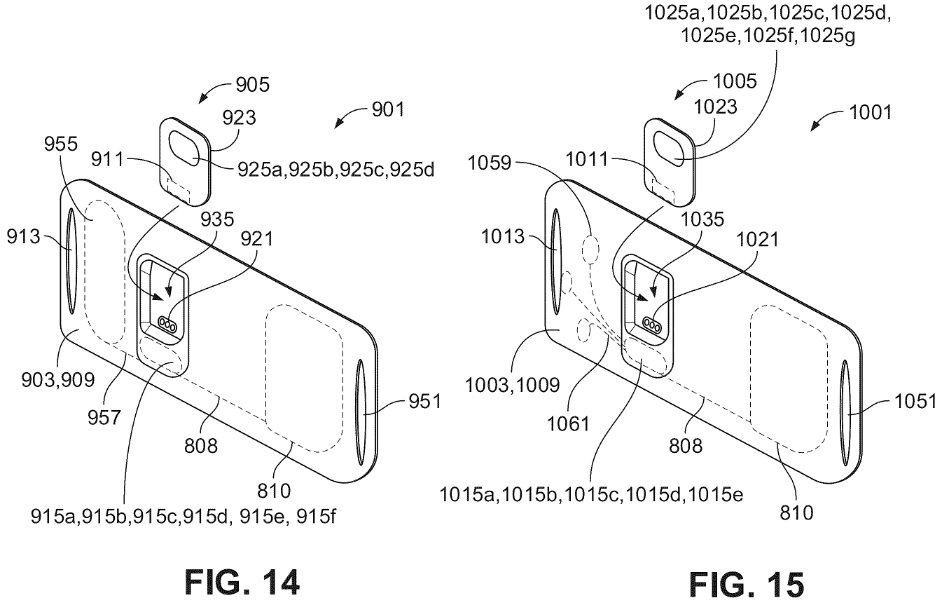

In some embodiments, a wearable assembly that is similar in construction and function to the wearable assembly 701 includes an additional embedded antenna for monitoring backscatter from the implanted tissue stimulator 814 of the tissue stimulation system 800. For example, FIG. 14 illustrates such a wearable assembly 901. The wearable assembly 901 includes a docking device 903 and a cooperating plug-in device 905 that can be attached to and removed from the docking device 903 as desired for administering treatment.

The docking device 903 includes a flexible fabric sleeve 909 that is substantially similar in construction and function to the sleeve 709 of the wearable assembly 701. The sleeve 909 is accordingly equipped with mating fastening features 913, 951 that are located along opposite edges of the sleeve 909. Within the sleeve 909, the docking device 903 further includes pulse generation functionalities of the RF generator module 806, the connection 808, and the TX antenna 810 of the tissue stimulation system 800, e.g, as embedded components. The sleeve also contains an additional embedded antenna 955, an additional embedded RF connector 957, and internal electronics 915a, 915b, 915c, 915d, 915c, 915f that support various functionalities of the wearable assembly 901. In some embodiments, the internal electronics may be formed on one or more flex circuits for imparting additional flexibility to the docking device 903. In some embodiments, the antennas and antenna connectors are discrete components that slip into pockets of the docking device 903, rather than being embedded in the sleeve 909.

The pulse generation functionalities of the RF generator module 806 are provided by the internal electronics 915a, 915b, which may be implemented respectively as an RF synthesizer and an RF gain amplifier for amplifying electrical pulses generated by the RF synthesizer. The antenna 955 functions as a receiver that monitors and measures backscatter from the implanted tissue stimulator 814. The internal electronics 915c, 915d therefore provide additional RF components and may be implemented as spectrum analyzer and a digital signal processor (DSP). The internal electronics 915e may be implemented as one or more power detectors for detecting power from the plug-in device 905, while the internal electronics 915f may be implemented as a non-volatile memory for storing unique patient data to allow the docking device 903 to be used interchangeably with multiple plug-in devices 905 that are associated with multiple patients.

The sleeve 909 is also equipped with a receptacle 935 that supports the plug-in device 905 at a connector terminal 921. The connection 808 of the tissue stimulation system 800 extends from the TX antenna 810 and terminates at the internal electronics 915a, 915b, 915c, 915d, 915c, 915f. The RF connector 957, extending from the antenna 955, also terminates at the internal electronics 915a, 915b, 915c, 915d, 915e, 915f. The connector terminal 921 is designed to mate with a connector terminal 911 of the plug-in device 905.

With the RF pulse generation functionalities of the RF pulse generator module 806 contained entirely within the docking device 903, the plug-in device 905 includes a power source of the RF pulse generator module 806. For example, in addition to the connector terminal 811, the plug-in device 905 also includes a housing 923 that contains internal electronics 925a. The internal electronics 925a are implemented as a battery for powering the internal electronics 915a, 915b, 915c, 915d, 915e, 915f within the docking device 903 and for powering additional internal electronics 925b, 925c, 925d contained within the housing 923. The internal electronics 925b, 925c, 925d may be implemented respectively as one or more processors, a UI controller, and the wireless connection 804 of the tissue stimulation system 800. Accordingly, the connector terminal 921 of the receptacle 935 provides power and data connections.

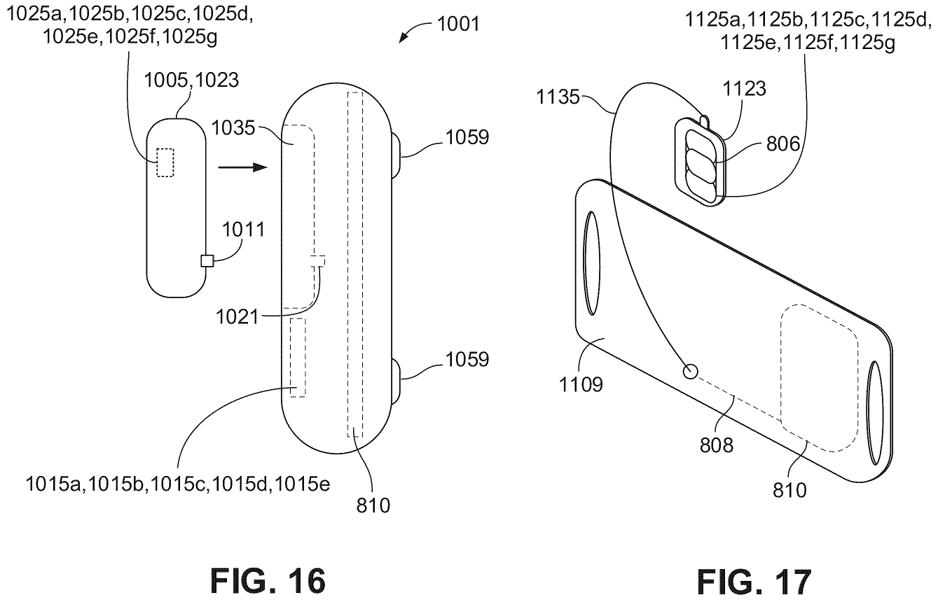

In some embodiments, a wearable assembly additionally includes skin contacting electrodes for sensing bioelectric signals. For example, FIGS. 15 and 16 illustrate such a wearable assembly 1001. The wearable assembly 1001 includes a docking device 1003 and a cooperating plug-in device 1005 that can be attached to and removed from the docking device 1003 as desired for administering treatment. The docking device 1003 includes a flexible fabric sleeve 1009 that is substantially similar in construction and function to the sleeve 709 of the wearable assembly 701. The sleeve 1009 is accordingly equipped with mating fastening features 1013, 1051 that are located along opposite edges of the sleeve 1009. Within the sleeve 1009, the docking device 1003 further includes the connection 808 and the TX antenna 810 of the tissue stimulation system 800, e.g., as embedded components.

Furthermore, the sleeve 1009 is equipped with skin contacting electrodes 1059 that can measure electrical potentials across the skin. The electrical potentials can be used to automatically turn on the pulse generator without user input, adjust a stimulation level, or to notify a user to manually adjust the stimulation level. For example, in some embodiments, the electrodes 1059 can sense a capacitive load to determine if the system is in contact with skin or not in contact with skin, such that the system may be controlled to turn on or off automatically. Such control can prevent the need for buttons. In some embodiments, the electrodes 1059 can sense electric potentials from the tissue stimulation to create a closed loop and adjust the tissue stimulation accordingly based on feedback from the sensors. In some embodiments, the electrodes 1059 can sense electric potentials and notify a user based on these potentials rather than autonomous efforts. In some embodiments, the electrodes 1059 can sense EKG or other health signals and store the signals as health data for further clinical study or use.

In some embodiments, information detected from the electrodes 1059 may be used for health tracking, such as PUSH-mosquito messaging (e.g., WiFi or IoT), health history of the patient for quick use of the wearable assembly 1001, failure prediction, recognition of a patient's therapy and geolocation, and smart home integration and voice activation. For example, health tracking may utilize data from multiple sources, such as the activity of sensors, a GPS location, and wireless communication modules to predict pain patterns and report such patterns to a user, a physician, a technician, the cloud, or artificial intelligence (AI). For instance, a patient's GPS tracker & accelerometer data may be processed by AI and recognize a reduced frequency in the patient leaving the house, such that AI may send a push notification to remind the patient to walk or exercise or automatically increase the amplitude to address an expected increase in pain.

In some embodiments, information detected from the electrodes 1059 may also be used for optimizing treatment parameters and/or battery life in order to strike a balance between treatment parameters and battery life.

The electrodes 1059 are secured to an exterior surface 1063 of the sleeve 1009, while the associated leads 1061 extend, internal to the sleeve 1009, from the electrodes 1059. In some embodiments, the electrodes 1059 may be provided as sticky pads made of one or both of gel and metal (e.g., gel-Ag/AgCl pads). In some embodiments, the electrodes 1059 may be made of one or more dry electrode materials, such as conductive textile, copper foil tape, flexible printed circuit (FPC), conductive rubber, silver-coated jersey-textile, stainless steel (e.g., 14301 alloy), silver (e.g., 925sterling silver), or stainless steel mesh.

In association with the electrodes 1059, the sleeve 1009 also contains internal electronics 1015a, 1015b, 1015c that may be implemented respectively as an instrument amplifier, an analog-to-digital (A/D) converter, a DSP processor, and memory. In some embodiments, the electrodes 1059 optionally have an additional transcutaneous stimulation capability, and the sleeve 1009 optionally includes internal electronics 1015d that may be implemented as a transcutaneous electrical nerve stimulation (TENS) pulse generator.

The sleeve 1009 also contains internal electronics 1015e that may be implemented as a non-volatile memory for storing unique patient data for multiple patients to allow the docking device 1003 to be used interchangeably with multiple plug-in devices 1005 that are associated with multiple patients. The sleeve 1009 is equipped with a receptacle 1035 that supports the plug-in device 1005 at a connector terminal 1021. The connection 808 of the tissue stimulation system 800 extends from the TX antenna 810 and terminates at the internal electronics 1015a, 1015b, 1015c, 1015d, 1015e. The leads 1061 also terminate at the internal electronics 1015a, 1015b, 1015c, 1015d, 1015e. The connector terminal 1021 is designed to mate with a connector terminal 1011 of the plug-in device 1005.

In the wearable assembly 1001, the RF pulse generator module 806 is contained entirely within the plug-in device 1005. For example, in addition to the connector terminal 1011, the plug-in device 1005 also includes a housing 1023 that contains internal electronics 1025a, 1025b, 1025c, 1025d, 1025e, 1025f, 1025g. The internal electronics 1025a, 1025b, 1025c are implemented respectively as an RF synthesizer, an RF gain amplifier, and a battery for powering the RF pulse generation functionalities. The internal electronics 1025c also power additional internal electronics 1025d, 1025e, 1025f, 1025g contained within the housing 1023. The internal electronics 1025d, 1025e, 1025f, 1025g may be implemented respectively as one or more processors, a UI controller, the wireless connection 804 of the tissue stimulation system 800, and one or more power detectors. Accordingly, the connector terminal 1021 of the receptacle 1035 provides RF and data connections.

In some embodiments, the RF generator module 806 of the tissue stimulation system 800 may be attached to a flexible fabric sleeve 1109 with a connecting cable 1135 and without a docking receptacle, as shown in FIG. 17. The connecting cable 1135 is attached to the connection 808 of the tissue stimulation system 800, which is included as an embedded component in the sleeve 1109 along with the TX antenna 810 of the tissue stimulation system 800. In alternative embodiments, the connection 808 and the connecting cable 1135 may be provided as a single cable. In yet other embodiments, a wireless connection may be used instead, as described earlier. The RF generation module 806 includes a housing 1123 that contains internal electronics 1125a, 1125b, 1125c, 1125d, 1125e, 1125f, 1125g that may be implemented respectively as a battery, one or more processors, a UI controller, a wireless communication module, an RF synthesizer, an RF gain amplifier, and one or more power detectors.

While the wearable assemblies 701, 801, 901, 1001, 1101 have been described and illustrated with respect to certain dimensions, sizes, shapes, arrangements, and materials, in some embodiments, a wearable assembly that is otherwise substantially similar in construction and function to any of the wearable assemblies 701, 801, 901, 1001, 1101 may include one or more different dimensions, sizes, shapes, arrangements, and materials. For example, while the sleeves 709, 809, 909, 1009, 1109 have been described as being wrapped snuggly around a patient's body part, in some embodiments, a wearable assembly that is otherwise substantially similar in construction and function to any of the wearable assemblies 701, 801, 901, 1001, 1101 may be alternatively embedded within (e.g., sewn or otherwise coupled to) an article of clothing that is worn snuggly against the patient's body.

In some embodiments, the sleeves 709, 809, 909, 1009, 1109 have a length in a range of about 13 cm to about 40 cm and a height in a range of about 1 cm to about 15 cm. In some embodiments, the wearable assemblies 701, 801, 901, 1001, 1101 each have a total weight in a range of about 0.1 kg to about 2 kg.

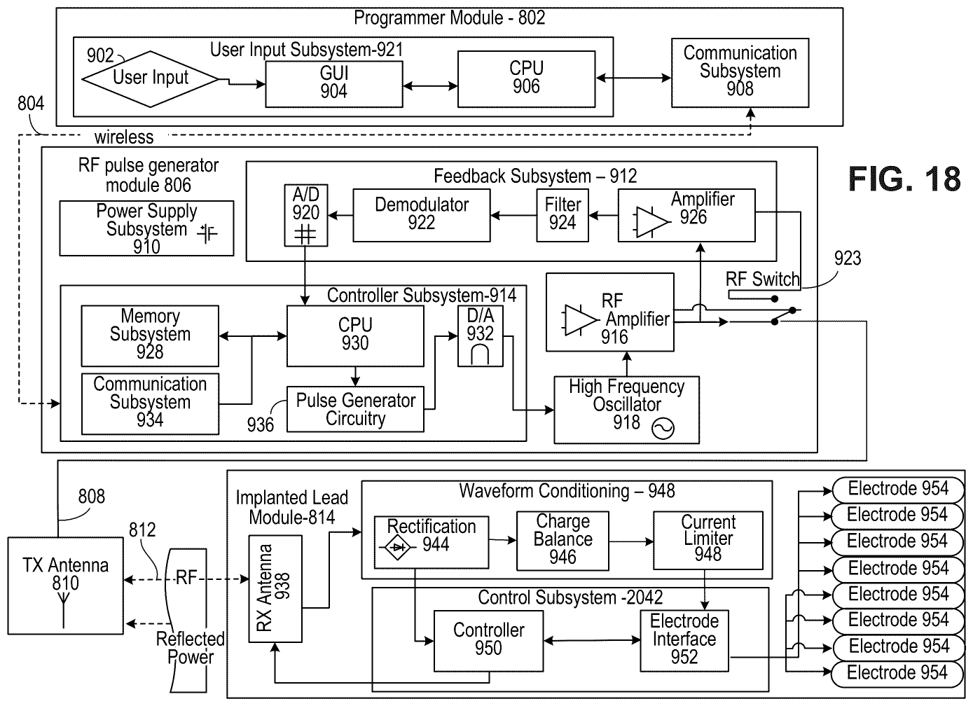

FIG. 18 depicts a detailed diagram of the tissue stimulation system 800. The programmer module 802 may be used as a vehicle to handle touchscreen input on a graphical user interface (GUI) 904 and may include a central processing unit (CPU) 906 for processing and storing data. The programmer module 802 includes a user input system 921 and a communication subsystem 908. The user input system 921 can allow a user to input or adjust instruction sets in order to adjust various parameter settings (e.g., in some cases, in an open loop fashion). The communication subsystem 908 can transmit these instruction sets (e.g., and other information) via the wireless connection 804 (e.g., via a Bluetooth or Wi-Fi connection) to the RF pulse generator module 806 (e.g., to the wearable module 100). The communication subsystem 908 can also receive data from RF pulse generator module 806.

The programmer module 802 can be utilized by multiple types of users (e.g., patients and others), such that the programmer module 802 may serve as a patient's control unit or a clinician's programmer unit. The programmer module 802 can be used to send stimulation parameters to the RF pulse generator module 806. The stimulation parameters that can be controlled may include a pulse amplitude in a range of 0 mA to 20mA, a pulse frequency in a range of 0 Hz to 2000 Hz, and a pulse width in a range of 0ms to 2 ms. In this context, the term pulse refers to the phase of the waveform that directly produces stimulation of the tissue. Parameters of a charge-balancing phase (described below) of the waveform can similarly be controlled. The user can also optionally control an overall duration and a pattern of a treatment.

The tissue stimulator 814 or the RF pulse generator module 806 may be initially programmed to meet specific parameter settings for each individual patient during an initial implantation procedure. Because medical conditions or the body itself can change over time, the ability to adjust the parameter settings may be beneficial to ensure ongoing efficacy of the neural modulation therapy.

Signals sent by the RF pulse generator module 806 to the tissue stimulator 814 may include both power and parameter attributes related to the stimulus waveform, amplitude, pulse width, and frequency. The RF pulse generator module 806 can also function as a wireless receiving unit that receives feedback signals from the tissue stimulator 814. To that end, the RF pulse generator module 806 includes microelectronics or other circuitry to handle the generation of the signals transmitted to the tissue stimulator 814, as well as feedback signals received from tissue stimulator 814. For example, the RF pulse generator module 806 includes a controller subsystem 914, a high-frequency oscillator 918, an RF amplifier 916, an RF switch, and a feedback subsystem 912.

The controller subsystem 914 includes a CPU 930 to handle data processing, a memory subsystem 928 (e.g., a local memory), a communication subsystem 934 to communicate with the programmer module 802 (e.g., including receiving stimulation parameters from the programmer module 802), pulse generator circuitry 936, and digital/analog (D/A) converters 932.

The controller subsystem 914 may be used by the user to control the stimulation parameter settings (e.g., by controlling the parameters of the signal sent from RF pulse generator module 806 to tissue stimulator 814). These parameter settings can affect the power, current level, or shape of the electrical pulses that will be applied by the electrodes. The programming of the stimulation parameters can be performed using the programming module 802 as described above to set a repetition rate, pulse width, amplitude, and waveform that will be transmitted by RF energy to a receive (RX) antenna 938 (e.g., or multiple RX antennas 938) within the tissue stimulator 814. The RX antenna 938 may be a dipole antenna or another type of antenna. A clinician user may have the option of locking and/or hiding certain settings within a programmer interface to limit an ability of a patient user to view or adjust certain parameters since adjustment of certain parameters may require detailed medical knowledge of neurophysiology, neuroanatomy, protocols for neural modulation, and safety limits of electrical stimulation.

The controller subsystem 914 may store received parameter settings in the local memory subsystem 928 until the parameter settings are modified by new input data received from the programmer module 802. The CPU 906 may use the parameters stored in the local memory to control the pulse generator circuitry 936 to generate a stimulus waveform that is modulated by the high frequency oscillator 918 in a range of 300 MHZ to 8 GHz. The resulting RF signal may then be amplified by an RF amplifier 926 and sent through an RF switch 923 to the TX antenna 810 to reach the RX antenna 938 through a depth of tissue.

In some implementations, the RF signal sent by the TX antenna 810 may simply be a power transmission signal used by tissue stimulator 814 to generate electric pulses. In other implementations, the RF signal sent by the TX antenna 810 may be a telemetry signal that provides instructions about various operations of the tissue stimulator 814. The telemetry signal may be sent by the modulation of the carrier signal through the skin. The telemetry signal is used to modulate the carrier signal (e.g., a high frequency signal) that is coupled to the antenna 938 and does not interfere with the input received on the same lead to power the tissue stimulator 814. In some embodiments, the telemetry signal and the powering signal are combined into one signal, where the RF telemetry signal is used to modulate the RF powering signal such that the tissue stimulator 814 is powered directly by the received telemetry signal. Separate subsystems in the tissue stimulator 814 harness the power contained in the signal and interpret the data content of the signal.

The RF switch 923 may be a multipurpose device (e.g., a dual directional coupler) that passes the relatively high amplitude, extremely short duration RF pulse to the TX antenna 810 with minimal insertion loss, while simultaneously providing two low-level outputs to the feedback subsystem 912. One output delivers a forward power signal to the feedback subsystem 912, where the forward power signal is an attenuated version of the RF pulse sent to the TX antenna 810, and the other output delivers a reverse power signal to a different port of the feedback subsystem 912, where reverse power is an attenuated version of the reflected RF energy from the TX Antenna 810.

During the on-cycle time (e.g., while an RF signal is being transmitted to tissue stimulator 814), the RF switch 923 is set to send the forward power signal to feedback subsystem 912. During the off-cycle time (e.g., while an RF signal is not being transmitted to the tissue stimulator 814), the RF switch 923 can change to a receiving mode in which the reflected RF energy and/or RF signals from the tissue stimulator 814 are received to be analyzed in the feedback subsystem 912.

The feedback subsystem 912 of the RF pulse generator module 806 may include reception circuitry to receive and extract telemetry or other feedback signals from tissue stimulator 814 and/or reflected RF energy from the signal sent by TX antenna 810. The feedback subsystem 912 may include an amplifier 926, a filter 924, a demodulator 922, and an A/D converter 920. The feedback subsystem 912 receives the forward power signal and converts this high-frequency AC signal to a DC level that can be sampled and sent to the controller subsystem 914. In this way, the characteristics of the generated RF pulse can be compared to a reference signal within the controller subsystem 914. If a disparity (e.g., an error) exists in any parameter, the controller subsystem 914 can adjust the output to the RF pulse generator 806. The nature of the adjustment can be proportional to the computed error. The controller subsystem 914 can incorporate additional inputs and limits on its adjustment scheme, such as the signal amplitude of the reverse power and any predetermined maximum or minimum values for various pulse parameters.

The reverse power signal can be used to detect fault conditions in the RF-power delivery system. In an ideal condition, when TX antenna 810 has perfectly matched impedance to the tissue that it contacts, the electromagnetic waves generated from the RF pulse generator module 806 pass unimpeded from the TX antenna 810 into the body tissue. However, in real-world applications, a large degree of variability exists in the body types of users, types of clothing worn, and positioning of the antenna 810 relative to the body surface. Since the impedance of the antenna 810 depends on the relative permittivity of the underlying tissue and any intervening materials and on an overall separation distance of the antenna 810 from the skin, there can be an impedance mismatch at the interface of the TX antenna 810 with the body surface in any given application. When such a mismatch occurs, the electromagnetic waves sent from the RF pulse generator module 806 are partially reflected at this interface, and this reflected energy propagates backward through the antenna feed.

The dual directional coupler RF switch 923 may prevent the reflected RF energy propagating back into the amplifier 926, and may attenuate this reflected RF signal and send the attenuated signal as the reverse power signal to the feedback subsystem 912. The feedback subsystem 912 can convert this high-frequency AC signal to a DC level that can be sampled and sent to the controller subsystem 914. The controller subsystem 914 can then calculate the ratio of the amplitude of the reverse power signal to the amplitude of the forward power signal. The ratio of the amplitude of reverse power signal to the amplitude level of forward power may indicate severity of the impedance mismatch.

In order to sense impedance mismatch conditions, the controller subsystem 914 can measure the reflected-power ratio in real time, and according to preset thresholds for this measurement, the controller subsystem 914 can modify the level of RF power generated by the RF pulse generator module 806. For example, for a moderate degree of reflected power the course of action can be for the controller subsystem 914 to increase the amplitude of RF power sent to the TX antenna 810, as would be needed to compensate for slightly non-optimum but acceptable TX antenna coupling to the body. For higher ratios of reflected power, the course of action can be to prevent operation of the RF pulse generator module 806 and set a fault code to indicate that the TX antenna 810 has little or no coupling with the body. This type of reflected power fault condition can also be generated by a poor or broken connection to the TX antenna 810. In either case, it may be desirable to stop RF transmission when the reflected power ratio is above a defined threshold, because internally reflected power can lead to unwanted heating of internal components, and this fault condition means that the system cannot deliver sufficient power to the tissue stimulator 814 and thus cannot deliver therapy to the user.

The controller 942 of the tissue stimulator 814 may transmit informational signals, such as a telemetry signal, through the RX antenna 538 to communicate with the RF pulse generator module 806 during its receive cycle. For example, the telemetry signal from the tissue stimulator 814 may be coupled to the modulated signal on the RX antenna 938, during the on and off state of the transistor circuit to enable or disable a waveform that produces the corresponding RF bursts necessary to transmit to the external (or remotely implanted) pulse generator module 806. The RX antenna 938 may be connected to electrodes 954 in contact with tissue to provide a return path for the transmitted signal. An A/D converter can be used to transfer stored data to a serialized pattern that can be transmitted on the pulse modulated signal from the RX antenna 938 of the tissue stimulator 814.

A telemetry signal from the tissue stimulator 814 may include stimulus parameters, such as the power or the amplitude of the current that is delivered to the tissue from the electrodes 954. The feedback signal can be transmitted to the RF pulse generator module 806 to indicate the strength of the stimulus at the target nerve tissue by means of coupling the signal to the RX antenna 938, which radiates the telemetry signal to the RF pulse generator module 806. The feedback signal can include either or both an analog and digital telemetry pulse modulated carrier signal. Data such as stimulation pulse parameters and measured characteristics of stimulator performance can be stored in an internal memory device within the tissue stimulator 814 and sent on the telemetry signal. The frequency of the carrier signal may be in a range of 300 MHz to 8 GHZ.

In the feedback subsystem 912, the telemetry signal can be down modulated using the demodulator 922 and digitized by being processed through the analog to digital (A/D) converter 920. The digital telemetry signal may then be routed to the CPU 930 with embedded code, with the option to reprogram, to translate the signal into a corresponding current measurement in the tissue based on the amplitude of the received signal. The CPU 930 of the controller subsystem 914 can compare the reported stimulus parameters to those held in local memory 928 to verify that the tissue stimulator 814 delivered the specified stimuli to target nerve tissue. For example, if the tissue stimulator 814 reports a lower current than was specified, the power level from the RF pulse generator module 806 can be increased so that the tissue stimulator 814 will have more available power for stimulation. The tissue stimulator 814 can generate telemetry data in real time (e.g., at a rate of 8 kbits per second). All feedback data received from the tissue stimulator 814 can be logged against time and sampled to be stored for retrieval to a remote monitoring system accessible by a health care professional for trending and statistical correlations.

The sequence of remotely programmable RF signals received by the RX antenna 938 may be conditioned into waveforms that are controlled within the tissue stimulator 814 by the control subsystem 942 and routed to the appropriate electrodes 954 that are located in proximity to the target nerve tissue. For instance, the RF signal transmitted from the RF pulse generator module 806 may be received by RX antenna 938 and processed by circuitry, such as waveform conditioning circuitry 940, within the tissue stimulator 814 to be converted into electrical pulses applied to the electrodes 954 through an electrode interface 952. In some implementations, the tissue stimulator 814 includes between two to sixteen electrodes 954.

The waveform conditioning circuitry 940 may include a rectifier 944, which rectifies the signal received by the RX antenna 938. The rectified signal may be fed to the controller 942 for receiving encoded instructions from the RF pulse generator module 806. The rectifier signal may also be fed to a charge balance component 946 that is configured to create one or more electrical pulses such that the one or more electrical pulses result in a substantially zero net charge at the one or more electrodes 954 (that is, the pulses are charge balanced). The charge balanced pulses are passed through the current limiter 948 to the electrode interface 952, which applies the pulses to the electrodes 954 as appropriate.

The current limiter 948 ensures the current level of the pulses applied to the electrodes 954 is not above a threshold current level. In some implementations, an amplitude (for example, a current level, a voltage level, or a power level) of the received RF pulse directly determines the amplitude of the stimulus. In this case, it may be particularly beneficial to include current limiter 948 to prevent excessive current or charge being delivered through the electrodes 954, although the current limiter 548 may be used in other implementations where this is not the case. Generally, for a given electrode 954 having several square millimeters of surface area, it is the charge per phase that should be limited for safety (where the charge delivered by a stimulus phase is the integral of the current). But, in some cases, the limit can instead be placed on the current, where the maximum current multiplied by the maximum possible pulse duration is less than or equal to the maximum safe charge. More generally, the current limiter 948 acts as a charge limiter that limits a characteristic (for example, a current or duration) of the electrical pulses so that the charge per phase remains below a threshold level (typically, a safe-charge limit).

In the event the tissue stimulator 814 receives a “strong” pulse of RF power sufficient to generate a stimulus that would exceed the predetermined safe-charge limit, the current limiter 948 can automatically limit or “clip” the stimulus phase to maintain the total charge of the phase within the safety limit. The current limiter 948 may be a passive current limiting component that cuts the signal to the electrodes 954 once the safe current limit (the threshold current level) is reached. Alternatively, or additionally, the current limiter 948 may communicate with the electrode interface 952 to turn off all electrodes 954 to prevent tissue damaging current levels.

A clipping event may trigger a current limiter feedback control mode. The action of clipping may cause the controller to send a threshold power data signal to the RF pulse generator module 806. The feedback subsystem 912 detects the threshold power signal and demodulates the signal into data that is communicated to the controller subsystem 914. The controller subsystem 914 algorithms may act on this current-limiting condition by specifically reducing the RF power generated by the RF pulse generator module 806, or cutting the power completely. In this way, the RF pulse generator module 806 can reduce the RF power delivered to the body if the tissue stimulator 814 reports that it is receiving excess RF power.

The controller 950 may communicate with the electrode interface 952 to control various aspects of the electrode setup and pulses applied to the electrodes 954. The electrode interface 952 may act as a multiplex and control the polarity and switching of each of the electrodes 954. For instance, in some implementations, the tissue stimulator 814 has multiple electrodes 954 in contact with the target neural tissue, and for a given stimulus, the RF pulse generator module 806 can arbitrarily assign one or more electrodes to act as a stimulating electrode, to act as a return electrode, or to be inactive by communication of assignment sent wirelessly with the parameter instructions, which the controller 950 uses to set electrode interface 952 as appropriate. It may be physiologically advantageous to assign, for example, one or two electrodes 954 as stimulating electrodes and to assign all remaining electrodes 954 as return electrodes.

Also, in some implementations, for a given stimulus pulse, the controller 950 may control the electrode interface 952 to divide the current arbitrarily (or according to instructions from the RF pulse generator module 806) among the designated stimulating electrodes. This control over electrode assignment and current control can be advantageous because in practice the electrodes 954 may be spatially distributed along various neural structures, and through strategic selection of the stimulating electrode location and the proportion of current specified for each location, the aggregate current distribution on the target neural tissue can be modified to selectively activate specific neural targets. This strategy of current steering can improve the therapeutic effect for the patient.

In another implementation, the time course of stimuli may be arbitrarily manipulated. A given stimulus waveform may be initiated at a time T_start and terminated at a time T_final, and this time course may be synchronized across all stimulating and return electrodes. Furthermore, the frequency of repetition of this stimulus cycle may be synchronous for all of the electrodes 954. However, the controller 950, on its own or in response to instructions from the RF pulse generator module 806, can control electrode interface 952 to designate one or more subsets of electrodes to deliver stimulus waveforms with non-synchronous start and stop times, and the frequency of repetition of each stimulus cycle can be arbitrarily and independently specified.

For example, a tissue stimulator 814 having eight electrodes 954 may be configured to have a subset of five electrodes, called set A, and a subset of three electrodes, called set B. Set A may be configured to use two of its electrodes as stimulating electrodes, with the remainder being return electrodes. Set B may be configured to have just one stimulating electrode. The controller 950 could then specify that set A deliver a stimulus phase with 3 mA current for a duration of 200 us, followed by a 400 us charge-balancing phase. This stimulus cycle could be specified to repeat at a rate of 60 cycles per second. Then, for set B, the controller 950 could specify a stimulus phase with 1 mA current for duration of 500 us, followed by a 800 us charge-balancing phase. The repetition rate for the set B stimulus cycle can be set independently of set A (e.g., at 25 cycles per second). Or, if the controller 950 was configured to match the repetition rate for set B to that of set A, for such a case the controller 950 can specify the relative start times of the stimulus cycles to be coincident in time or to be arbitrarily offset from one another by some delay interval.

In some implementations, the controller 950 can arbitrarily shape the stimulus waveform amplitude, and may do so in response to instructions from the RF pulse generator module 806. The stimulus phase may be delivered by a constant-current source or a constant-voltage source, and this type of control may generate characteristic waveforms that are static. For example, a constant current source generates a characteristic rectangular pulse in which the current waveform has a very steep rise, a constant amplitude for the duration of the stimulus, and then a very steep return to baseline. Alternatively, or additionally, the controller 950 can increase or decrease the level of current at any time during the stimulus phase and/or during the charge-balancing phase. Thus, in some implementations, the controller 950 can deliver arbitrarily shaped stimulus waveforms such as a triangular pulse, sinusoidal pulse, or Gaussian pulse for example. Similarly, the charge-balancing phase can be arbitrarily amplitude-shaped, and similarly a leading anodic pulse (prior to the stimulus phase) may also be amplitude-shaped.

As described above, the tissue stimulator 814 may include a charge balancing component 946. Generally, for constant current stimulation pulses, pulses should be charge balanced by having the amount of cathodic current should equal the amount of anodic current, which is typically called biphasic stimulation. Charge density is the amount of current times the duration it is applied, and is typically expressed in the units uC/cm². In order to avoid the irreversible electrochemical reactions such as pH change, electrode dissolution as well as tissue destruction, no net charge should appear at the electrode-electrolyte interface, and it is generally acceptable to have a charge density less than 30 uC/cm². Biphasic stimulating current pulses ensure that no net charge appears at the electrode 954 after each stimulation cycle and that the electrochemical processes are balanced to prevent net de currents. The tissue stimulator 814 may be designed to ensure that the resulting stimulus waveform has a net zero charge. Charge balanced stimuli are thought to have minimal damaging effects on tissue by reducing or eliminating electrochemical reaction products created at the electrode-tissue interface.

A stimulus pulse may have a negative-voltage or current, called the cathodic phase of the waveform. Stimulating electrodes may have both cathodic and anodic phases at different times during the stimulus cycle. An electrode 954 that delivers a negative current with sufficient amplitude to stimulate adjacent neural tissue is called a “stimulating electrode.” During the stimulus phase, the stimulating electrode acts as a current sink. One or more additional electrodes act as a current source and these electrodes are called “return electrodes.” Return electrodes are placed elsewhere in the tissue at some distance from the stimulating electrodes. When a typical negative stimulus phase is delivered to tissue at the stimulating electrode, the return electrode has a positive stimulus phase. During the subsequent charge-balancing phase, the polarities of each electrode are reversed.

In some implementations, the charge balance component 946 uses one or more blocking capacitors placed electrically in series with the stimulating electrodes and body tissue, between the point of stimulus generation within the stimulator circuitry and the point of stimulus delivery to tissue. In this manner, a resistor-capacitor (RC) network may be formed. In a multi-electrode stimulator, one charge-balance capacitors may be used for each electrode, or a centralized capacitors may be used within the stimulator circuitry prior to the point of electrode selection. The RC network can block direct current (DC). However, the RC network can also prevent low-frequency alternating current (AC) from passing to the tissue. The frequency below which the series RC network essentially blocks signals is commonly referred to as the cutoff frequency, and in some embodiments, the design of the stimulator system may ensure that the cutoff frequency is not above the fundamental frequency of the stimulus waveform. In the example embodiment 800, the tissue stimulator 814 may have a charge-balance capacitor with a value chosen according to the measured series resistance of the electrodes and the tissue environment in which the stimulator is implanted. By selecting a specific capacitance value, the cutoff frequency of the RC network in this embodiment is at or below the fundamental frequency of the stimulus pulse.

In other implementations, the cutoff frequency may be chosen to be at or above the fundamental frequency of the stimulus, and in this scenario the stimulus waveform created prior to the charge-balance capacitor, called the drive waveform, may be designed to be non-stationary, where the envelope of the drive waveform is varied during the duration of the drive pulse. For example, in one embodiment, the initial amplitude of the drive waveform is set at an initial amplitude Vi, and the amplitude is increased during the duration of the pulse until it reaches a final value k*Vi. By changing the amplitude of the drive waveform over time, the shape of the stimulus waveform passed through the charge-balance capacitor is also modified. The shape of the stimulus waveform may be modified in this fashion to create a physiologically advantageous stimulus.

In some implementations, the tissue stimulator 814 may create a drive-waveform envelope that follows the envelope of the RF pulse received by the RX antenna 938. In this case, the RF pulse generator module 806 can directly control the envelope of the drive waveform within the tissue stimulator 814, and thus no energy storage may be required inside of the tissue stimulator 814, itself. In this implementation, the stimulator circuitry may modify the envelope of the drive waveform or may pass it directly to the charge-balance capacitor and/or electrode-selection stage.

In some implementations, the tissue stimulator 814 may deliver a single-phase drive waveform to the charge balance capacitor or it may deliver multiphase drive waveforms. In the case of a single-phase drive waveform (e.g., a negative-going rectangular pulse), this pulse comprises the physiological stimulus phase, and the charge-balance capacitor is polarized (charged) during this phase. After the drive pulse is completed, the charge balancing function is performed entirely by the passive discharge of the charge-balance capacitor, where is dissipates its charge through the tissue in an opposite polarity relative to the preceding stimulus. In one implementation, a resistor within the tissue stimulator 814 facilitates the discharge of the charge-balance capacitor. In some implementations, using a passive discharge phase, the capacitor may allow virtually complete discharge prior to the onset of the subsequent stimulus pulse.

In the case of multiphase drive waveforms, the tissue stimulator 814 may perform internal switching to pass negative-going or positive-going pulses (phases) to the charge-balance capacitor. These pulses may be delivered in any sequence and with varying amplitudes and waveform shapes to achieve a desired physiological effect. For example, the stimulus phase may be followed by an actively driven charge-balancing phase, and/or the stimulus phase may be preceded by an opposite phase. Preceding the stimulus with an opposite-polarity phase, for example, can have the advantage of reducing the amplitude of the stimulus phase required to excite tissue.

In some implementations, the amplitude and timing of stimulus and charge-balancing phases is controlled by the amplitude and timing of RF pulses from the RF pulse generator module 806, and in other implementations, this control may be administered internally by circuitry onboard the tissue stimulator 814, such as controller 550. In the case of onboard control, the amplitude and timing may be specified or modified by data commands delivered from the pulse generator module 806.

While the RF pulse generator module 806 and the TX antenna 810 have been described and illustrated as separate components, in some embodiments, the RF pulse generator module 806 and the TX antenna 810 may be physically located in the same housing or other packaging. Furthermore, while the RF pulse generator module 806 and the TX antenna 810 have been described and illustrated as located external to the body, in some embodiments, either or both of the RF pulse generator module 806 and the TX antenna 810 may be designed to be implanted subcutaneously. While the RF pulse generator module 806 and the TX antenna 810 have been described and illustrated as coupled via a wired connection 808, in some embodiments (e.g., where the RF pulse generator module 806 is either located externally or implanted subcutaneously), the RF pulse generator module 806 and the TX antenna 810 may be coupled via a wireless connection.

While the tissue stimulation system 800 has been described and illustrated with respect to certain dimensions, sizes, shapes, arrangements, and materials, in some embodiments, a tissue stimulation system that is otherwise substantially similar in construction and function to the tissue stimulation system 800 may include one or more different dimensions, sizes, shapes, arrangements, and materials. Accordingly, other embodiments are also within the scope of the following claims.

What is claimed is:

1. A wearable assembly configured to generate electrical pulses for transmission to an implanted tissue stimulator, the wearable assembly comprising:
a flexible band configured to be worn by a patient having the implanted tissue stimulator;
a docking connection receptacle integral to the flexible band, wherein the docking connection receptacle is equipped with a docking connector terminal that is integral to the docking connection receptacle;
a transmitting antenna disposed within the flexible band and electrically coupled by wires to the docking connection receptacle so as to receive electrical signals through the docking connection receptacle and transmit one or more electromagnetic signals upon receipt of such electrical signals to the implanted tissue stimulator; and
a portable pulse generation device configured to be removably secured within the docking connection receptacle of the flexible band, the portable pulse generation device configured to output electrical signals to a device connector terminal of the portable pulse generation device, wherein securing the portable pulse generation device within the docking connection receptacle results in the device connector terminal mating with the docking connector terminal so the electrical signals from the portable pulse generation device pass to the transmitting antenna for transmission to the implanted tissue stimulator.

2. The wearable assembly of claim 1, wherein the transmitting antenna is integral with the flexible band.

3. The wearable assembly of claim 1, wherein the flexible band is configured with opposite edges and wherein mating fastenings are located on the opposite edges of the flexible band so that the flexible band can be wrapped about a body part and the opposite edges fastened together.

4. The wearable assembly of claim 1, wherein the flexible band is stretchable such that the flexible band expands when wrapped around a body of a user and when removed from the body, the flexible band returns to an original shape.

5. The wearable assembly of claim 1, further comprising one or more electronic components within and integral with the flexible band and electrically connected to the docking connector terminal.

6. The wearable assembly of claim 5, wherein the one or more electronic components is a non-volatile memory.

7. The wearable assembly of claim 6, wherein the non-volatile memory stores data relating to the portable pulse generation device that has been mated to the docking connector terminal.

8. The wearable assembly of claim 7, wherein the data stored by the non-volatile memory includes identification data that identifies a unique one of a plurality of portable pulse generation devices.

9. The wearable assembly of claim 8, where the data includes information relating to a plurality of unique portable pulse generation devices that have been mated to the docking connector terminal.

10. The wearable assembly of claim 1, further comprising a secondary antenna disposed within and integral with the flexible band and electrically coupled to the docking connection receptacle by one of a plurality of wires within the flexible band.

11. The wearable assembly of claim 10, wherein the secondary antenna is configured to receive signals and provide the received signals to a mated pulse generation device.

12. The wearable assembly of claim 1, wherein the portable pulse generation device is configured with a plurality of settings that include at least one of: an amplitude setting, a pulse width setting, a frequency setting, and a preset program setting.

13. The wearable assembly of claim 1, further including one or more skin contacting electrodes integral with the flexible band.

14. The wearable assembly of claim 13, wherein the one or more skin contacting electrodes are connected to the docking connection receptacle.

15. The wearable assembly of claim 13, wherein the portable pulse generation device is configured, after connection to the docking connection receptacle, to generate a plurality of pre-programmed pulses in response to the portable pulse generation device receiving a signal indicating that the skin contacting electrodes are contacting skin of a wearer.

16. The wearable assembly of claim 13, further comprising one or more internal electronic devices connected to the skin contacting electrodes and configured to process one or more signals generated by the skin contacting electrodes.

17. The wearable assembly of claim 16, wherein the process carried out by the one or more electronic devices is that of amplification, analog-to-digital conversion, and/or digital signal processing.

18. The wearable assembly of claim 16, wherein the internal electronic devices are configured as an electrical nerve stimulation (TENS) pulse generator.

19. A wearable assembly configured to generate electrical pulses for transmission to an implanted tissue stimulator, the wearable assembly comprising:
a flexible band configured to be worn by a patient having the implanted tissue stimulator;
a docking connection receptacle integral to the flexible band, wherein the docking connection receptacle is equipped with a docking connector terminal that is integral to the docking connection receptacle;
a transmitting antenna secured to the flexible band and electrically coupled by wires to the docking connection receptacle so as to receive electrical signals through the docking connection receptacle and transmit one or more electromagnetic signals upon receipt of such electrical signals to the implanted tissue stimulator; and
a portable pulse generation device configured to be removably secured within the docking connection receptacle of the flexible band, the portable pulse generation device configured to exchange electrical signals through the docking connector terminal with the transmitting antenna.

20. A wearable assembly configured to generate electrical pulses for transmission to an implanted tissue stimulator, the wearable assembly comprising:
a flexible band configured to be worn by a patient having the implanted tissue stimulator;
a docking connection receptacle integral to the flexible band, wherein the docking connection receptacle is equipped with a docking connector terminal that is integral to the docking connection receptacle;
a transmitting antenna disposed within the flexible band and electrically coupled by wires to the docking connection receptacle so as to receive and transmit electrical signals through the docking connection receptacle; and
a portable pulse generation device configured to be removably secured within the docking connection receptacle of the flexible band, the portable pulse generation device configured to automatically output pre-programmed electrical signals to a device connector terminal of the portable pulse generation device when the portable pulse generation device is secured within the flexible band.
